(19) United States
(12) Patent Application Publication
(10) Pub. No.: US 2020/0037986 A1
(43) Pub. Date: Feb. 6, 2020

(54) METHODS AND APPARATUSES FOR GUIDING COLLECTION OF ULTRASOUND DATA USING MOTION AND/OR ORIENTATION DATA

(71) Applicant: Butterfly Network, Inc., Guilford, CT (US)

(72) Inventors: Nathan Silberman, Brooklyn, NY (US); Tomer Gafner, Forest Hills, NY (US); Igor Lovchinsky, New York, NY (US); Ardavan Saeedi, Jersey City, NJ (US)

(73) Assignee: Butterfly Network, Inc., Guilford, CT (US)

(21) Appl. No.: 16/529,860

(22) Filed: Aug. 2, 2019

Related U.S. Application Data
(60) Provisional application No. 62/714,620, filed on Aug. 3, 2018.

(51) Int. Cl. A61B 8/00; G06N 3/08

(52) U.S. Cl. CPC A61B 8/4254; G06N 3/08; A61B 8/46

(57) ABSTRACT

Aspects of the technology described herein relate to guiding collection of ultrasound data collection using motion and/or orientation data. Motion and/or orientation data may be received from an ultrasound imaging device, where the motion and/or orientation data provides an indication of the motion and/or orientation of the ultrasound imaging device. Ultrasound data collected by the ultrasound imaging device may also be received. An instruction for moving the ultrasound imaging device based on the motion and/or orientation data and the ultrasound data may be provided. Ultrasound data and motion and/or orientation data may indicate a velocity of the ultrasound imaging device that exceeds a threshold velocity and an instruction for slowing the velocity of the ultrasound imaging device may be provided.

### CROSS-REFERENCE TO RELATED APPLICATIONS

The present application claims the benefit under 35 USC § 119(e) of U.S. patent application Ser. No. 62/714,620, filed Aug. 3, 2018, under Attorney Docket No. B1348.70081US00, and entitled “METHODS AND APPARATUSES FOR GUIDING COLLECTION OF ULTRASOUND DATA USING MOTION AND/OR ORIENTATION DATA,” which is hereby incorporated herein by reference in its entirety.

### FIELD

Generally, the aspects of the technology described herein relate to ultrasound data collection. Some aspects relate to guiding collection of ultrasound data using motion and/or orientation data from an ultrasound imaging device.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using an ultrasound imaging device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method includes receiving, by a processing device, motion and/or orientation data from an ultrasound imaging device in operative communication with the processing device, wherein the motion and/or orientation data provides an indication of a motion and/or orientation of the ultrasound imaging device; receiving, by the processing device, ultrasound data collected by the ultrasound imaging device; and providing, by the processing device, an instruction for moving the ultrasound imaging device based on the motion and/or orientation data and the ultrasound data.

In some embodiments, the instruction for moving the ultrasound imaging device includes an instruction for moving the ultrasound imaging device to a target position and orientation. In some embodiments, the motion and/or orientation data and the ultrasound data are associated with each other based on time. In some embodiments, the method further includes inputting the motion and/or orientation data and the ultrasound data to a statistical model configured to output the instruction for moving the ultrasound imaging device based on the motion and/or orientation data and the ultrasound data. In some embodiments, the inputted motion and/or orientation data and the inputted ultrasound data include first motion and/or orientation data and first ultrasound data; and the statistical model is configured to output the instruction for moving the ultrasound imaging device further based on second motion and/or orientation data and second ultrasound data, wherein the first motion and/or orientation data was received more recently than the second motion and/or orientation data and the first ultrasound data was received more recently than the second ultrasound data. In some embodiments, the statistical model includes a recurrent neural network. In some embodiments, the recurrent neural network includes a long short-term memory neural network. In some embodiments, the ultrasound imaging device is configured to generate the motion and/or orientation data using one or more of an accelerometer, a gyroscope, or a magnetometer on the ultrasound imaging device.

According to another aspect, a method includes receiving, by a processing device, sets of ultrasound data two or more times from an ultrasound imaging device in operative communication with the processing device; receiving, by the processing device, motion and/or orientation data from the ultrasound imaging device, wherein the motion and/or orientation data provides an indication of a motion and/or orientation of the ultrasound imaging device; determining that the sets of ultrasound data and the motion and/or orientation data indicate a velocity of the ultrasound imaging device that exceeds a threshold velocity; and based on determining that the sets of ultrasound data and the motion and/or orientation data indicate that the velocity of the ultrasound imaging device exceeds the threshold velocity, providing an instruction for slowing the velocity of the ultrasound imaging device.

In some embodiments, the processing device is configured to access a statistical model configured to output the velocity of the ultrasound imaging device based on the sets of ultrasound data and the motion and/or orientation data. In some embodiments, the threshold velocity is related to a lag time between when the ultrasound imaging device collects ultrasound data and when the processing device provides an instruction for moving the ultrasound imaging device to a target location and orientation based on the ultrasound data. In some embodiments, the processing device is configured to access a statistical model configured to output the instruction for moving the ultrasound imaging device to the target location and orientation based on the ultrasound data. In some embodiments, the ultrasound imaging device is configured to generate the motion and/or orientation data using one or more of an accelerometer, a gyroscope, or a magnetometer on the ultrasound imaging device. In some embodiments, the threshold velocity is approximately in the range of 0.25 cm/s-2 cm/s.

Some aspects include at least one non-transitory computer-readable storage medium storing processor-executable instructions that, when executed by at least one processor, cause the at least one processor to perform the above aspects and embodiments. Some aspects include an apparatus having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a schematic block diagram of an example ultrasound system upon which various aspects of the technology described herein may be practiced;

FIG. 2 illustrates an example process for guiding collection of ultrasound data using ultrasound, motion, and orientation data, in accordance with certain embodiments described herein;

FIG. 3 illustrates an example process for guiding collection of ultrasound data using ultrasound data and motion data, in accordance with certain embodiments described herein;

FIG. 4 illustrates an example process for guiding collection of ultrasound data using ultrasound data and orientation data, in accordance with certain embodiments described herein;

FIG. 5 illustrates an example process for guiding collection ultrasound data by determining whether an ultrasound imaging device exceeds a threshold velocity, in accordance with certain embodiments described herein;

FIG. 6 illustrates another example process for guiding collection ultrasound data by determining whether an ultrasound imaging device exceeds a threshold velocity, in accordance with certain embodiments described herein;

FIG. 7 illustrates another example process for guiding collection ultrasound data by determining whether an ultrasound imaging device exceeds a threshold velocity, in accordance with certain embodiments described herein; and

FIG. 8 illustrates an example convolutional neural network that is configured to analyze an image.

### DETAILED DESCRIPTION

Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and further how to properly position the ultrasound device on the subject to capture a medically relevant ultrasound image of the anatomical structure. Holding the ultrasound device a few inches too high or too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include capturing ultrasound images of the incorrect anatomical structure and capturing foreshortened (or truncated) ultrasound images of the correct anatomical structure.

Conventional ultrasound systems are large, complex, and expensive systems that are typically only purchased by large medical facilities with significant financial resources. Recently, cheaper and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application), which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound imaging devices makes them more accessible to the general populace, people who could make use of such devices have little to no training for how to use them. For example, a small clinic without a trained ultrasound technician on staff may purchase an ultrasound device to help diagnose patients. In this example, a nurse at the small clinic may be familiar with ultrasound technology and human physiology, but may know neither which anatomical views of a patient need to be imaged in order to identify medically-relevant information about the patient nor how to obtain such anatomical views using the ultrasound device. In another example, an ultrasound device may be issued to a patient by a physician for at-home use to monitor the patient's heart. In all likelihood, the patient understands neither human physiology nor how to image his or her own heart with the ultrasound device.

Accordingly, the inventors have developed assistive ultrasound imaging technology for guiding an operator of an ultrasound device how to move the ultrasound device relative to an anatomical area of a subject in order to capture medically relevant ultrasound data. The inventors have recognized that a statistical model may be configured to output an instruction for moving an ultrasound imaging device to a target position and/or orientation on a subject based on inputted motion and/or orientation data of the ultrasound imaging device and ultrasound data collected by the ultrasound imaging device. The motion and/or orientation data may include any data indicating motion and/or orientation of an object. For example, the motion and/or orientation data may include data regarding acceleration of the object, data regarding angular velocity of the object, and/or data regarding magnetic force acting on the object (which, due to the magnetic field of the earth, may be indicative of orientation relative to the earth). The acceleration data may be generated by an accelerometer, the angular velocity data may be generated by a gyroscope, and the magnetic force data may be generated by a magnetometer. Two or more of these sensors may be integrated into a single sensor device, such as an inertial measurement unit. Depending on the devices used to generate the motion and/or orientation data, the motion and/or orientation data may describe three degrees of freedom, six degrees of freedom, or nine degrees of freedom for the ultrasound imaging device.

The statistical model may be configured to output an instruction for moving the ultrasound imaging device to a target position and/or orientation based on the most recent motion and/or orientation data of the ultrasound imaging device and the most recent ultrasound data collected by the ultrasound imaging device at the most recent position/orientation, as well as based on previous motion and/or orientation data and ultrasound data that were inputted to the statistical model. This may be helpful, for example, if the motion and/or orientation data includes acceleration data, as the statistical model may be able to integrate the most recent acceleration data and previous acceleration data to determine information regarding the path in space previously traveled by the ultrasound imaging device. Comparing the current path traveled by the ultrasound imaging device to other paths traveled by ultrasound imaging devices used to generate training data may assist the statistical model in outputting more accurate instructions. The statistical model may also be able to rely upon motion and/or orientation data to generate instructions in cases in which the statistical model cannot determine information from ultrasound data (e.g., the ultrasound data is poor quality). Accordingly, the accuracy of the instructions produced by the statistical model may be increased by using inputs of associated motion and/or orientation data and ultrasound data, rather than just using ultrasound data as the input.

The inventors have also recognized that the velocity of an ultrasound imaging device may be determined from motion and/or orientation data. The inventors have recognized that providing instructions to a user to slow down movement of an ultrasound imaging device when the velocity of the ultrasound imaging device exceeds a threshold velocity may be helpful in providing more accurate instructions for moving the ultrasound imaging device. For example, a statistical model may accept ultrasound data as an input and output an instruction for moving the ultrasound imaging device to a target position and/or orientation based on the ultrasound data. The threshold velocity may be related to the lag time between when the ultrasound imaging device collects ultrasound data and when the processing device provides the instruction. As another example, if the statistical model has not been trained on sequences of ultrasound images collected by ultrasound imaging devices moving beyond the threshold velocity, the statistical model may not provide accurate instructions based on ultrasound images collected by an ultrasound imaging device moving beyond the threshold velocity. Providing instructions to a user to slow down movement of the ultrasound imaging device may help to increase the accuracy of instructions provided by the statistical model. As another example, moving an ultrasound imaging device too fast may result in blurry ultrasound images, and providing instructions to a user to slow down movement of the ultrasound imaging device may help to improve the quality of ultrasound images collected.

Various aspects of the present application are described as providing or implementing statistical models. In some embodiments, a statistical model may be a convolutional neural network having one or more convolutional layers, a recurrent neural network, a fully-connected neural network, and/or any other suitable type of deep neural network model, a random forest, a support vector machine, a linear classifier, a Bayesian classifier, a non-parametric statistical model, and/or any other statistical model unless otherwise noted.

As referred to herein, a device displaying an item (e.g., a directional indicator on an augmented reality display) should be understood to mean that the device displays the item on the device's own display screen, or generates the item to be displayed on another device's display screen. To perform the latter, the device may transmit instructions to the other device for displaying the item.

As referred to herein, an augmented reality display should be understood to mean any display superimposing non-real two- or three-dimensional graphics on images/video of the real three-dimensional world such that the two- or three-dimensional graphics appear to be present in the three-dimensional world.

As referred to herein, any action performed based on some input criterion/criteria should be understood to mean that the action is performed based solely on the input criterion/criteria or based on the input criterion/criteria and other input criterion/criteria. For example, a determination made based on ultrasound data should be understood to mean that the determination is either made based on the ultrasound data or based on the ultrasound data and other input data.

As referred to herein, a first device that is in operative communication with a second device should be understood to mean that the first device may transmit signals to the second device and thereby affect operation of the second device. The second device may also transmit signals to the first device and thereby affect operation of the first device.

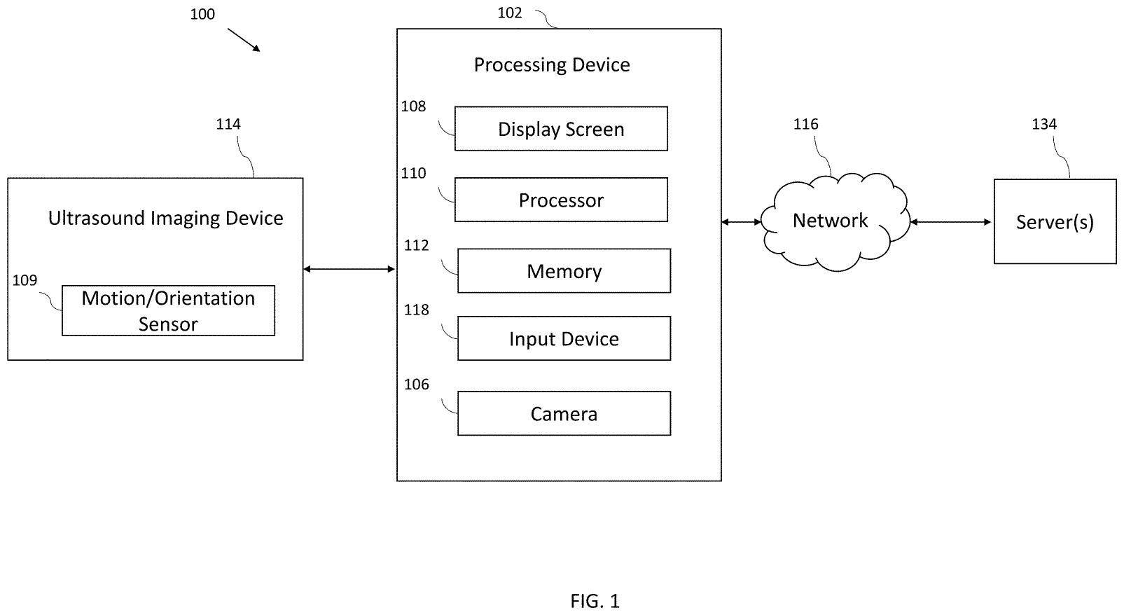

FIG. 1 illustrates a schematic block diagram of an example ultrasound system 100 upon which various aspects of the technology described herein may be practiced. The ultrasound system 100 includes an ultrasound imaging device 114, a processing device 102, a network 116, and one or more servers 134.

The ultrasound imaging device 114 includes a motion and/or orientation sensor 109. The processing device 102 includes a camera 106, a display screen 108, a processor 110, a memory 112, an input device 118, and a motion and/or orientation sensor 109. The processing device 102 is in wired (e.g., through a lightning connector or a mini-USB connector) and/or wireless communication (e.g., using BLUETOOTH, ZIGBEE, and/or WiFi wireless protocols) with the ultrasound imaging device 114. The processing device 102 is in wireless communication with the one or more servers 134 over the network 116.

The ultrasound imaging device 114 may be configured to generate ultrasound data that may be employed to generate an ultrasound image. The ultrasound imaging device 114 may be constructed in any of a variety of ways. In some embodiments, the ultrasound imaging device 114 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound imaging device 114 may include one or more ultrasonic transducers monolithically integrated onto a single semiconductor die. The ultrasonic transducers may include, for example, one or more capacitive micromachined ultrasonic transducers (CMUTs), one or more piezoelectric micromachined ultrasonic transducers (PMUTs), and/or one or more other suitable ultrasonic transducer cells. In some embodiments, the ultrasonic transducers may be formed from or on the same chip as other electronic components (e.g., transmit circuitry, receive circuitry, control circuitry, power management circuitry, and processing circuitry) to form a monolithic ultrasound device. The ultrasound imaging device 114 may transmit ultrasound data and/or ultrasound images to the processing device 102 over a wired (e.g., through a lightning connector or a mini-USB connector) and/or wireless (e.g., using BLUETOOTH, ZIGBEE, and/or WiFi wireless protocols) communication link.

The motion and/or orientation sensor 109 may be configured to generate motion and/or orientation data regarding the ultrasound imaging device 114. For example, the motion and/or orientation sensor 109 may be configured to generate data regarding acceleration of the ultrasound imaging device 114, data regarding angular velocity of the ultrasound imaging device 114, and/or data regarding magnetic force acting on the ultrasound imaging device 114 (which, due to the magnetic field of the earth, may be indicative of orientation relative to the earth). The motion and/or orientation sensor 109 may include an accelerometer, a gyroscope, and/or a magnetometer. Depending on the sensors present in the motion and/or orientation sensor 109, the motion and/or orientation data generated by the motion and/or orientation sensor 109 may describe three degrees of freedom, six degrees of freedom, or nine degrees of freedom for the ultrasound imaging device 114. For example, the motion and/or orientation sensor 109 may include an accelerometer, a gyroscope, and/or magnetometer. Each of these types of sensors may describe three degrees of freedom. If the motion and/or orientation sensor 109 includes one of these sensors, the motion and/or orientation sensor 109 may describe three degrees of freedom. If the motion and/or orientation sensor 109 includes two of these sensors, the motion and/or orientation sensor 109 may describe two degrees of freedom. If the motion and/or orientation sensor 109 includes three of these sensors, the motion and/or orientation sensor 109 may describe nine degrees of freedom. The ultrasound imaging device 114 may transmit motion and/or orientation data to the processing device 102 over a wired (e.g., through a lightning connector or a mini-USB connector) and/or wireless (e.g., using BLUETOOTH, ZIGBEE, and/or WiFi wireless protocols) communication link.

Referring now to the processing device 102, the processor 110 may include specially-programmed and/or special-purpose hardware such as an application-specific integrated circuit (ASIC). For example, the processor 110 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 102 may be configured to process the ultrasound data received from the ultrasound imaging device 114 to generate ultrasound images for display on the display screen 108. The processing may be performed by, for example, the processor 110. The processor 110 may also be adapted to control the acquisition of ultrasound data with the ultrasound imaging device 114. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 102 may be configured to perform certain of the processes described herein using the processor 110 (e.g., one or more computer hardware processors) and one or more articles of manufacture that include non-transitory computer-readable storage media such as the memory 112. The processor 110 may control writing data to and reading data from the memory 112 in any suitable manner. To perform certain of the processes described herein, the processor 110 may execute one or more processor-executable instructions stored in one or more non-transitory computer-readable storage media (e.g., the memory 112), which may serve as non-transitory computer-readable storage media storing processor-executable instructions for execution by the processor 110. The camera 106 may be configured to detect light (e.g., visible light) to form an image. The display screen 108 may be configured to display images and/or videos, and may be, for example, a liquid crystal display (LCD), a plasma display, and/or an organic light emitting diode (OLED) display on the processing device 102. The input device 118 may include one or more devices capable of receiving input from a user and transmitting the input to the processor 110. For example, the input device 118 may include a keyboard, a mouse, a microphone, touch-enabled sensors on the display screen 108, and/or a microphone. The display screen 108, the input device 118, the camera 106, and the speaker 109 may be communicatively coupled to the processor 110 and/or under the control of the processor 110.

It should be appreciated that the processing device 102 may be implemented in any of a variety of ways. For example, the processing device 102 may be implemented as a handheld device such as a mobile smartphone or a tablet. Thereby, a user of the ultrasound imaging device 114 may be able to operate the ultrasound imaging device 114 with one hand and hold the processing device 102 with another hand. In other examples, the processing device 102 may be implemented as a portable device that is not a handheld device, such as a laptop. In yet other examples, the processing device 102 may be implemented as a stationary device such as a desktop computer. The processing device 102 may be connected to the network 116 over a wired connection (e.g., via an Ethernet cable) and/or a wireless connection (e.g., over a WiFi network). The processing device 102 may thereby communicate with (e.g., transmit data to) the one or more servers 134 over the network 116. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application).

FIG. 1 should be understood to be non-limiting. For example, the ultrasound system 100 may include fewer or more components than shown and the processing device 102 may include fewer or more components than shown.

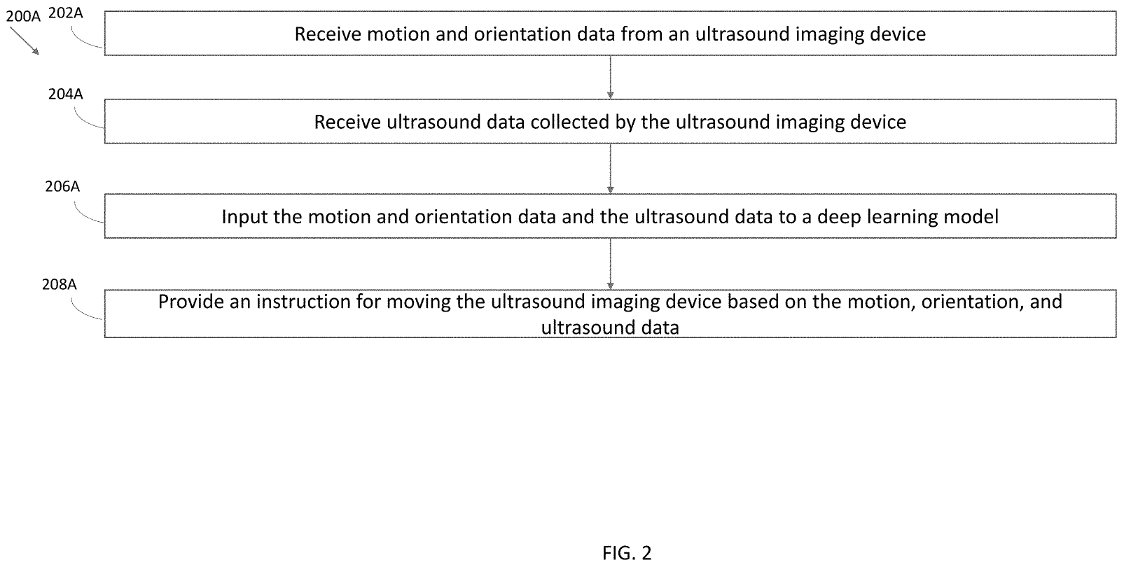

FIG. 2 illustrates an example process 200A for guiding collection of ultrasound data using ultrasound data, motion data, and orientation data, in accordance with certain embodiments described herein. The process 200A may be performed by a processing device (e.g., processing device 102) in an ultrasound system (e.g., ultrasound system 100). The processing device may be, for example, a mobile phone, tablet, laptop, or server, and may be in operative communication with an ultrasound imaging device (e.g., ultrasound imaging device 114).

In act 202A, the processing device receives motion and orientation data from an ultrasound imaging device. For example, the motion and orientation data may include data regarding acceleration of an object (e.g., an ultrasound probe having ultrasound transducer elements), data regarding angular velocity of the object, and/or data regarding magnetic force acting on the object (which, due to the magnetic field of the earth, may be indicative of orientation relative to the earth). The ultrasound imaging device may include an accelerometer, a gyroscope, and/or a magnetometer, and these devices may be used by the ultrasound imaging device to generate the motion and orientation data. Depending on the devices used to generate the motion and orientation data, the motion and orientation data may describe three degrees of freedom, six degrees of freedom, or nine degrees of freedom for the ultrasound imaging device. The ultrasound imaging device may transmit the motion and orientation data over a wired communication link (e.g., over Ethernet, a Universal Serial Bus (USB) cable or a Lightning cable) or over a wireless communication link (e.g., over a BLUETOOTH, WiFi, or ZIGBEE wireless communication link) to the processing device. The process 200A proceeds from act 202A to act 204A.

In act 204A, the processing device receives ultrasound data collected by the ultrasound imaging device. The ultrasound data may include, for example, raw acoustical data, scan lines generated from raw acoustical data, or ultrasound images generated from raw acoustical data. In some embodiments, the ultrasound imaging device may generate scan lines and/or ultrasound images from raw acoustical data and transmit the scan lines and/or ultrasound images to the processing device. In other embodiments, the ultrasound imaging device may transmit the raw acoustical data to the processing device and the processing device may generate the scan lines and/or ultrasound images from the raw acoustical data. In still other embodiments, the ultrasound imaging device may generate scan lines from the raw acoustical data, transmit the scan lines to the processing device, and the processing device may generate ultrasound images from the scan lines. The ultrasound imaging device may transmit the ultrasound data over a wired communication link (e.g., over Ethernet, a Universal Serial Bus (USB) cable or a Lightning cable) or over a wireless communication link (e.g., over a BLUETOOTH, WiFi, or ZIGBEE wireless communication link) to the processing device. The ultrasound data received in act 204A and the motion and orientation data received in act 202A may be associated with each other based on time. For example, motion and orientation data regarding the motion and orientation of the ultrasound imaging device at a particular time or within a particular time period may be associated with ultrasound data collected by the ultrasound imaging device at the particular time or within the particular time period. In some embodiments, to associate the ultrasound data and the motion and orientation data with each other, the ultrasound data and the motion and orientation data may be associated with corresponding timestamps. The process 200A proceeds from act 204A to act 206A. It should be appreciated that while FIG. 2 depicts act 202A as occurring before act 204A, in some embodiments act 202A may occur after or simultaneously with act 204A.

In act 206A, the processing device inputs the motion and orientation data received in act 202A and the ultrasound data received in act 204A to a statistical model. In act 208A, the processing device provides an instruction for moving the ultrasound imaging device based on the motion and orientation data and the ultrasound data. In embodiments in which the ultrasound data and the motion and orientation data are associated with each other based on time, the processing device may input the associated motion and orientation data and ultrasound data to the statistical model together. In some embodiments, the ultrasound data and motion and orientation may be appended together and inputted to the statistical model. In some embodiments, the ultrasound data may be inputted to the statistical model independently of the motion and orientation data, and the statistical model may be structured to fuse the different types of data together after they have been independently processed by the statistical model. The statistical model may be configured to accept associated motion and/or orientation data and ultrasound data and output an instruction for moving the ultrasound imaging device based on the motion and/or orientation data and ultrasound data. The instruction may include an instruction for moving the ultrasound imaging device to a target position and/or orientation (e.g., relative to a subject being imaged) and may include any combination of instructions to translate, rotate, and tilt the ultrasound imaging device. The target position and/or orientation of the ultrasound imaging device may be a position and/or orientation of the ultrasound imaging device relative to a subject such that the ultrasound imaging device can collect a target anatomical view (e.g., a parasternal long axis view of the heart).

In some embodiments, the process 200A may be repeated until the ultrasound imaging device has been moved to the target position and/or orientation. In particular, after a user moves the ultrasound imaging device to a new position and/or orientation in response to an instruction, the processing device may receive new motion and orientation data and ultrasound data from the new position/orientation of the ultrasound imaging device and provide a new instruction for moving the ultrasound imaging device. Each instruction may be intended to instruct the user to move the ultrasound imaging device closer to the target position and/or orientation until the user has moved the ultrasound imaging device to the target position and/or orientation. For example, the target position and/or orientation of the ultrasound imaging device may be a position and/or orientation of the ultrasound imaging device relative to the subject at which the ultrasound imaging device can collect a parasternal long axis view of the heart. The processing device may first provide instructions to translate the ultrasound imaging device to a position at the cardiac region of the subject, and then provide instructions to rotate and/or tilt the ultrasound imaging at that position until the particular parasternal long axis view of the heart can be collected.

In some embodiments, the statistical model may be configured, for example through training, to accept associated motion and/or orientation data and ultrasound data and output an instruction for moving the ultrasound imaging device to a target position/orientation based on the motion and/or orientation data and ultrasound data. In particular, the statistical model may be trained on sets of training data, where each set of training data includes motion and/or orientation data for an ultrasound imaging device at a particular position/orientation relative to a subject, ultrasound data collected from the subject when the ultrasound imaging device is at the particular position/orientation relative to the subject, and a label indicating an instruction for moving the ultrasound imaging device from the particular position/orientation to the target position/orientation. Annotations of ultrasound data/images by a doctor, sonographer, or other medical professional may be used to generate the labels indicating the instructions. The statistical model may thereby learn what instruction to provide based on inputted motion and/or orientation data and ultrasound data.

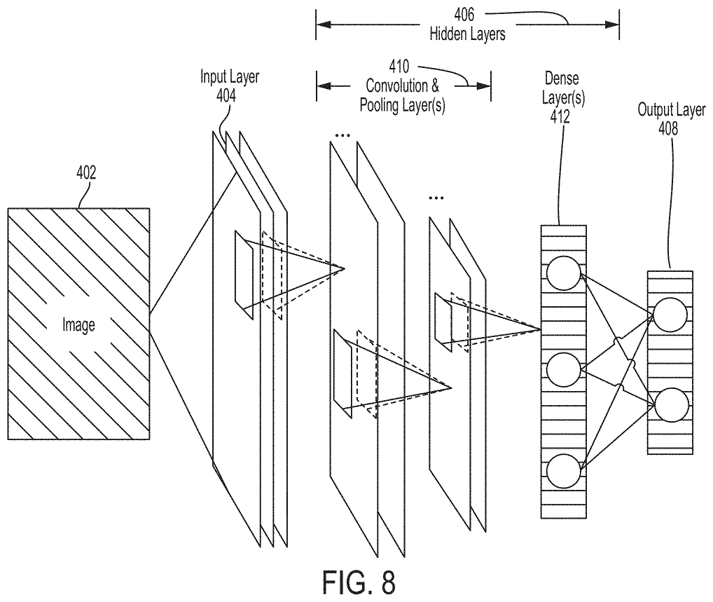

The statistical model may be a convolutional neural network, a random forest, a support vector machine, a linear classifier, and/or any other statistical model. The statistical model used may operate using memory. For example, the statistical model may be a recurrent neural network (e.g., a long short-term memory neural network). The statistical model may be configured to output an instruction for moving the ultrasound imaging device to a target position/orientation based on the most recent motion and/or orientation data for the ultrasound imaging device and the most recent ultrasound data collected by the ultrasound imaging device at the most recent position/orientation, as well as based on motion and/or orientation and ultrasound data from previous positions/orientations that was inputted to the statistical model. This may be helpful, for example, if the motion and/or orientation data includes acceleration data, as the statistical model may be able to integrate the most recent acceleration data and previous acceleration data to determine information regarding the path in space previously traveled by the ultrasound imaging device. Comparing the current path traveled by the ultrasound imaging device to other paths traveled by ultrasound imaging devices used to generate training data may assist the statistical model in outputting more accurate instructions. In some embodiments, the processing device may input the most recent acceleration data and previous acceleration data to the statistical model and the statistical model may integrate the inputted acceleration data to determine information regarding the path in space previously traveled by the ultrasound imaging device. In some embodiments, the processing device may integrate the most recent acceleration data and previous acceleration data and input the integrated acceleration data to the statistical model. The statistical model may also be able to rely upon motion and/or orientation data to generate instructions in cases in which the statistical model cannot determine information from ultrasound data (e.g., the ultrasound data is poor quality). Accordingly, the accuracy of the instructions produced by the statistical model may be increased by using inputs of associated motion and/or orientation data and ultrasound data, rather than just using ultrasound data as the input. For further description of statistical models and techniques, see the description with reference to FIG. 8.

In some embodiments, the statistical model may be stored in memory on the processing device and accessed internally by the processing device. In other embodiments, the statistical model may be stored in memory on another device, such as a remote server, and the processing device may transmit the motion and orientation data and the ultrasound data to the external device. The external device may input the motion and orientation data and ultrasound data to the statistical model and transmit the instruction outputted by the statistical model back to the processing device. Transmission between the processing device and the external device may be over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

To provide the instruction for moving the ultrasound imaging device, the processing device may display the instruction on a display screen (e.g., display screen 108) of the processing device. For example, if the processing device is a smartphone coupled to the ultrasound imaging device by a cable, the smartphone may display the instruction on its display screen. The displayed instruction may include any combination of words (e.g., “Move the probe medially,” “Rotate the probe clockwise”) and directional indicators (e.g., arrows). The processing device may display directional indicators on an image of a person. In some embodiments, the processing device may receive or capture real-time video of the subject and display directional indicators superimposed on the video of the subject in real-time, where the direction of the directional indicators indicates the direction in which the ultrasound imaging device should be moved. This may be considered an augmented reality display. In some embodiments, as an addition or alternative to providing visual instructions and indicators, the processing device may generate audio containing the instructions from speakers (e.g., speakers included in the processing device).

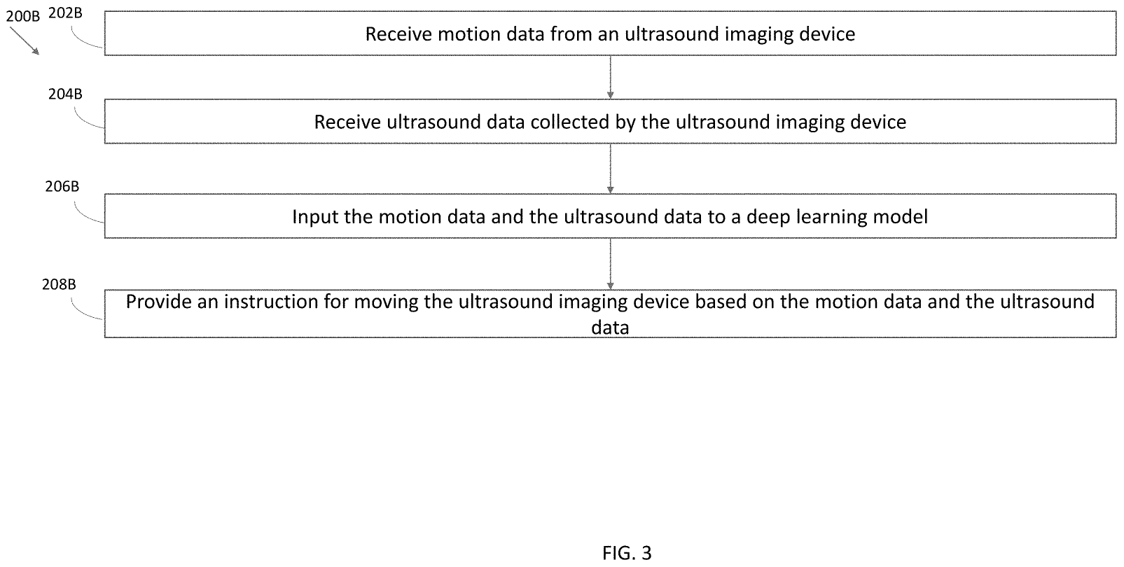

FIG. 3 illustrates an example process 200B for guiding collection of ultrasound data using ultrasound data and motion data, in accordance with certain embodiments described herein. The process 200B is similar to the process 200A, except that orientation data is not used. Any other aspect of process 200A may apply to process 200B as well.

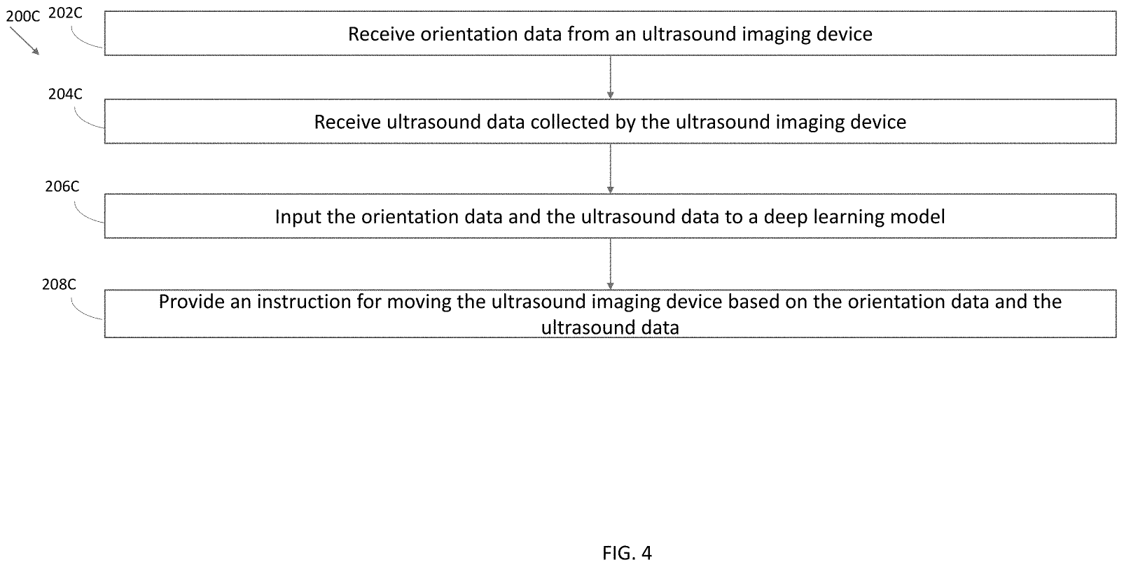

FIG. 4 illustrates an example process 200C for guiding collection of ultrasound data using ultrasound data and orientation data, in accordance with certain embodiments described herein. The process 200C is similar to the process 200A, except that the motion data is not used. Any other aspect of process 200A may apply to process 200C as well.

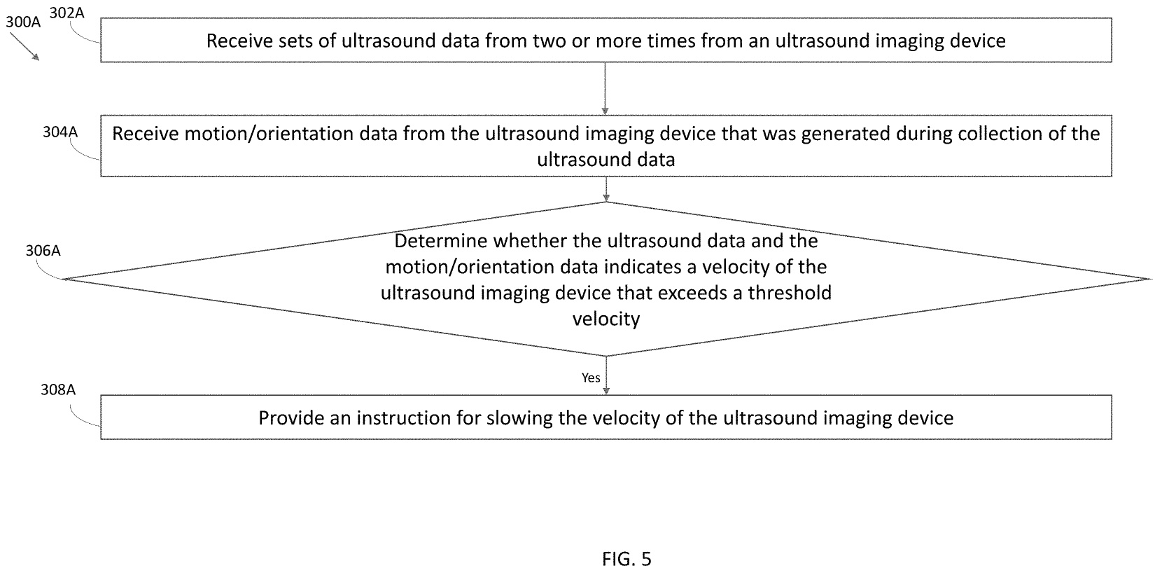

FIG. 5 illustrates an example process 300A for guiding collection of ultrasound data by determining whether an ultrasound imaging device exceeds a threshold velocity, in accordance with certain embodiments described herein. The process 300A may be performed by a processing device (e.g., processing device 102) in an ultrasound system (e.g., ultrasound system 100). The processing device may be, for example, a mobile phone, tablet, laptop, or server, and may be in operative communication with an ultrasound imaging device (e.g., ultrasound imaging device 114).

In act 302A, the processing devices receives sets of ultrasound data from two or more times from an ultrasound imaging device. For example, the ultrasound data may include a set of ultrasound data collected at one time from one location on a subject and a set of ultrasound data collected at a later time from another location on a subject. The ultrasound data may include, for example, raw acoustical data, scan lines generated from raw acoustical data, or ultrasound images generated from raw acoustical data. In some embodiments, the ultrasound imaging device may generate scan lines and/or ultrasound images from raw acoustical data and transmit the scan lines and/or ultrasound images to the processing device. In other embodiments, the ultrasound imaging device may transmit the raw acoustical data to the processing device and the processing device may generate the scan lines and/or ultrasound images from the raw acoustical data. In still other embodiments, the ultrasound imaging device may generate scan lines from the raw acoustical data, transmit the scan lines to the processing device, and the processing device may generate ultrasound images from the scan lines. The ultrasound imaging device may transmit the ultrasound data over a wired communication link (e.g., over Ethernet, a Universal Serial Bus (USB) cable or a Lightning cable) or over a wireless communication link (e.g., over a BLUETOOTH, WiFi, or ZIGBEE wireless communication link) to the processing device. The process 300A proceeds from act 302A to act 304A.

In act 304A, the processing device receives motion and/or orientation data from the ultrasound imaging device that was generated during collection of the ultrasound data in act 302A. For example, the motion and/or orientation data may include data regarding acceleration of the object, data regarding angular velocity of the object, and/or data regarding magnetic force acting on the object (which, due to the magnetic field of the earth, may be indicative of orientation relative to the earth). The ultrasound imaging device may include an accelerometer, a gyroscope, and/or a magnetometer, and these devices may be used by the ultrasound imaging device to generate the motion and/or orientation data. Depending on the devices used to generate the motion and/or orientation data, the motion and/or orientation data may describe three degrees of freedom, six degrees of freedom, or nine degrees of freedom for the ultrasound imaging device. The ultrasound imaging device may transmit the motion and/or orientation data over a wired communication link (e.g., over Ethernet, a Universal Serial Bus (USB) cable or a Lightning cable) or over a wireless communication link (e.g., over a BLUETOOTH, WiFi, or ZIGBEE wireless communication link) to the processing device. The process 300A proceeds from act 304A to act 306A.

In act 306A, the processing device determines whether the ultrasound data received in act 302A and the motion and/or orientation data received in act 304A indicates a velocity of the ultrasound imaging device that exceeds a threshold velocity. If the processing device determines that the velocity of the ultrasound imaging device exceeds the threshold velocity, the process 300A proceeds from act 306A to act 308A. In act 308A, the processing device provides an instruction to the user for slowing the velocity of the ultrasound imaging device. In some embodiments, the processing device may be configured to access a statistical model configured to accept, as inputs, ultrasound data from two or more times collected by an ultrasound imaging device and motion and/or orientation data for the ultrasound imaging device generated during collection of the ultrasound data, and output a velocity of the ultrasound imaging device. To train the statistical model to determine velocity from ultrasound data, the statistical model may be trained on ultrasound data, each set of which is labeled with the time when the ultrasound data was collected and the position of the ultrasound imaging device when it collected the ultrasound data. The statistical model may be able to determine the velocity of the ultrasound imaging device during collection of two sets of ultrasound data based on differences in the position and time corresponding to each set of ultrasound data. For example, if one set of ultrasound data was collected at position p1 and time t1 and another set of ultrasound data was collected at position p2 and time t2, the statistical model may determine the velocity of the ultrasound imaging device during collection of the two sets of ultrasound data to be (p1−p2)/(t1−t2). In embodiments in which the motion and/or orientation data includes acceleration data for the ultrasound imaging device, the statistical model may be able to determine the velocity of the ultrasound imaging device by integrating the acceleration data. The statistical model may be able to more accurately determine the velocity of the ultrasound imaging device using both ultrasound data and motion and/or orientation data. In some embodiments, the statistical model may determine the velocity of the ultrasound imaging device based only on ultrasound data. In such embodiments, act 304A may be absent. In some embodiments, the statistical model may determine the velocity of the ultrasound imaging device based only on motion and/or orientation data. In such embodiments, act 302A may be absent.

In some embodiments, the processing device may be configured to access another statistical model configured to accept ultrasound data as an input and output an instruction for moving the ultrasound imaging device to a target position and/or orientation based on the ultrasound data. In such embodiments, the processing device may be configured to provide the instruction. The threshold velocity may be related to the lag time between when the ultrasound imaging device collects ultrasound data and when the processing device provides the instruction. In some embodiments, the threshold velocity may be approximately in the range of 0.25 cm/s-2 cm/s, such as 0.25 cm/s, 0.5 cm/s, 0.75 cm/s, 1 cm/s, 1.25 cm/s, 1.5 cm/s, 1.75 cm/s, 2 cm/s, or any other suitable threshold velocity. The inventors have recognized that providing instructions to a user to slow down movement of an ultrasound imaging device when the velocity of the ultrasound imaging device exceeds a threshold velocity may be helpful in providing more accurate instructions for moving the ultrasound imaging device. As another example, if the statistical model has not been trained on sequences of ultrasound images collected by ultrasound imaging devices moving beyond the threshold velocity, the statistical model may not provide accurate instructions based on ultrasound images collected by an ultrasound imaging device moving beyond the threshold velocity. Providing instructions to a user to slow down movement of the ultrasound imaging device may help to increase the accuracy of instructions provided by the statistical model. As another example, moving an ultrasound imaging device too fast may result in blurry ultrasound images, and providing instructions to a user to slow down movement of the ultrasound imaging device may help to improve the quality of ultrasound images collected.

To provide the instruction for slowing the velocity of the ultrasound imaging device, the processing device may display the instruction on a display screen (e.g., display screen 108) of the processing device. For example, if the processing device is a smartphone coupled to the ultrasound imaging device by a cable, the smartphone may display the instruction on its display screen. The displayed instruction may include words (e.g., “Slow down”). In some embodiments, the processing device may generate audio containing the instructions from speakers (e.g., speakers included in the processing device). The instruction provided in act 308A may be provided in conjunction with the instruction provided in acts 208A, 208B, and/or 208C. For example, when a user moves the ultrasound imaging device in response to the instruction provided in acts 208A, 208B, and/or 208C, if the user moves the ultrasound imaging device too fast, the instruction of act 308A may be provided to slow down movement of the ultrasound imaging device.

In some embodiments, the processing device may determine whether ultrasound data and motion and/or orientation data indicates a velocity of the ultrasound imaging device that is less than a threshold velocity, and if so, provide an instruction to speed up movement of the ultrasound imaging device. This may be helpful if the statistical model has not been trained on sequences of ultrasound images collected by ultrasound imaging devices moving below the threshold velocity, as the statistical model may not provide accurate instructions based on ultrasound images collected by an ultrasound imaging device moving below the threshold velocity. Providing instructions to a user to speed up movement of the ultrasound imaging device may help to increase the accuracy of instructions provided by the statistical model.

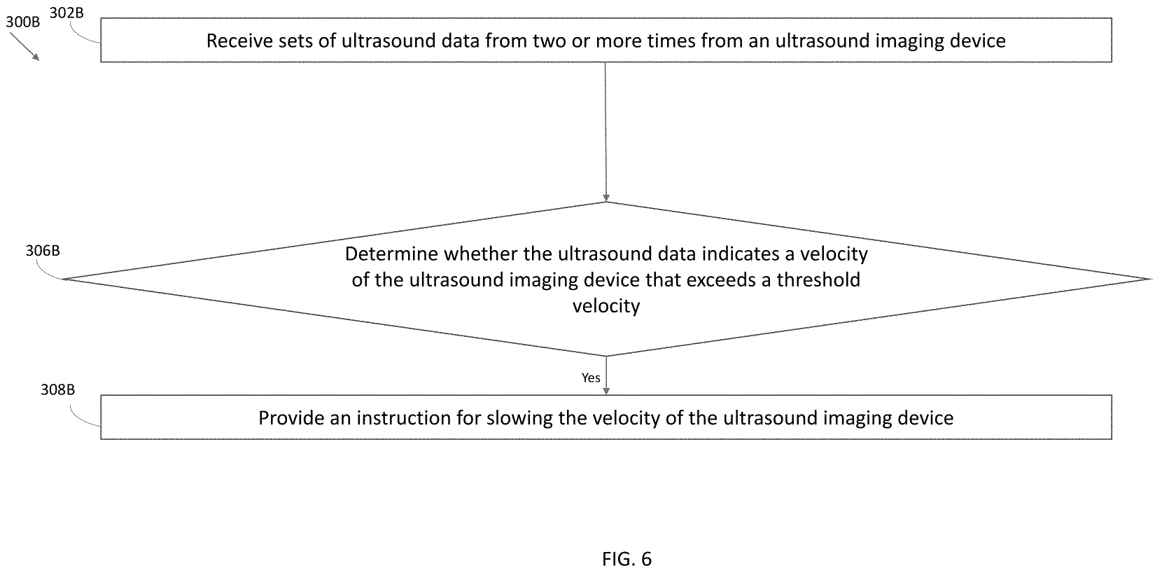

FIG. 6 illustrates another example process 300B for guiding collection of ultrasound data by determining whether an ultrasound imaging device exceeds a threshold velocity, in accordance with certain embodiments described herein. The process 300B is similar to the process 300A, except that motion/orientation data is not used. All other aspects of the process 300A may apply to the process 300B.

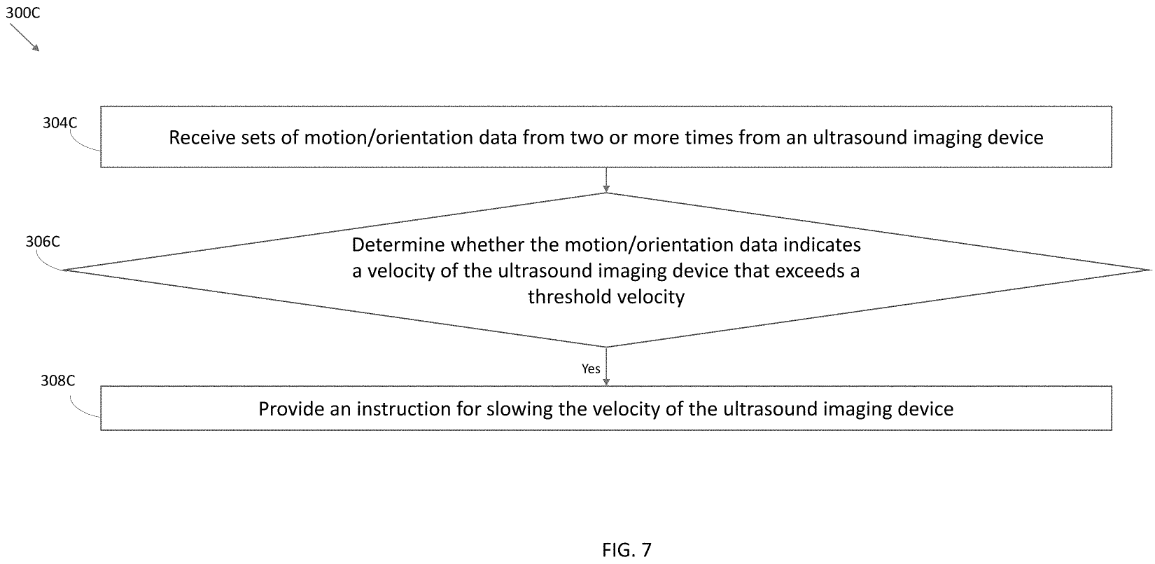

FIG. 7 illustrates another example process 300C for guiding collection of ultrasound data by determining whether an ultrasound imaging device exceeds a threshold velocity, in accordance with certain embodiments described herein. The process 300C is similar to the process 300A, except that ultrasound data is not used. All other aspects of the process 300A may apply to the process 300C.

The above description has described the processes 200A-C and 300A-C as being performed by a processing device in operative communication with an ultrasound imaging device. However, it should be appreciated that any steps of the processes 200A-C and 300A-C may also be performed by the ultrasound imaging device itself or any combination of devices in operative communication with the ultrasound imaging device and each other. For example, when the process is performed by the ultrasound imaging device 114 itself, the ultrasound imaging device 114 may include the processor 110, the memory 112, the display screen 108, the input device 118, and/or the camera 106. The processor 110 of the ultrasound imaging device 114 may execute one or more processor-executable instructions stored in one or more non-transitory computer-readable storage media (e.g., the memory 112 of the ultrasound imaging device 114), which may serve as non-transitory computer-readable storage media storing processor-executable instructions for execution by the processor 110. Additionally, the embodiments described herein may also be applied to ultrasound devices used for other purposes besides imaging, such as ultrasound devices for treatment (e.g., high-intensity focused ultrasound (HIFU)).

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

Aspects of the technology described herein relate to the application of automated image processing techniques to analyze images, such as ultrasound images. In some embodiments, the automated image processing techniques may include machine learning techniques such as statistical techniques. Machine learning techniques may include techniques that seek to identify patterns in a set of data points and use the identified patterns to make predictions for new data points. These machine learning techniques may involve training (and/or building) a model using a training data set to make such predictions.

Statistical techniques may include those machine learning techniques that employ neural networks to make predictions. Neural networks typically include a collection of neural units (referred to as neurons) that each may be configured to receive one or more inputs and provide an output that is a function of the input. For example, the neuron may sum the inputs and apply a transfer function (sometimes referred to as an “activation function”) to the summed inputs to generate the output. The neuron may apply a weight to each input, for example, to weight some inputs higher than others. Example transfer functions that may be employed include step functions, piecewise linear functions, rectified linear unit (ReLu) functions, and sigmoid functions. These neurons may be organized into a plurality of sequential layers that each include one or more neurons. The plurality of sequential layers may include an input layer that receives the input data for the neural network, an output layer that provides the output data for the neural network, and one or more hidden layers connected between the input and output layers. Each neuron in a hidden layer may receive inputs from one or more neurons in a previous layer (such as the input layer) and provide an output to one or more neurons in a subsequent layer (such as an output layer).

A neural network may be trained using, for example, labeled training data. The labeled training data may include a set of example inputs and an answer associated with each input. For example, the training data may include a plurality of ultrasound images or sets of raw acoustical data that are each labeled with an instruction for moving an ultrasound imaging device from the position/orientation where the inputted ultrasound data was collected to a target position/orientation. In this example, the ultrasound images may be provided to the neural network to obtain outputs that may be compared with the labels associated with each of the ultrasound images. One or more characteristics of the neural network (such as the interconnections between neurons (referred to as edges) in different layers and/or the weights associated with the edges) may be adjusted until the neural network correctly classifies most (or all) of the input images.

Once the training data has been created, the training data may be loaded to a database (e.g., an image database) and used to train a neural network using statistical techniques. Once the neural network has been trained, the trained neural network may be deployed to one or more processing devices.

In some applications, a neural network may be implemented using one or more convolution layers to form a convolutional neural network. An example convolutional neural network is shown in FIG. 8 that is configured to analyze an image 402. As shown, the convolutional neural network includes an input layer 404 to receive the image 402, an output layer 408 to provide the output, and a plurality of hidden layers 406 connected between the input layer 404 and the output layer 408. The plurality of hidden layers 406 includes convolution and pooling layers 410 and dense layers 412.

The input layer 404 may receive the input to the convolutional neural network. As shown in FIG. 8, the input the convolutional neural network may be the image 402. The image 402 may be, for example, an ultrasound image.

The input layer 404 may be followed by one or more convolution and pooling layers 410. A convolutional layer may include a set of filters that are spatially smaller (e.g., have a smaller width and/or height) than the input to the convolutional layer (e.g., the image 402). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 410 may be followed by dense layers 412. The dense layers 412 may include one or more layers each with one or more neurons that receives an input from a previous layer (e.g., a convolutional or pooling layer) and provides an output to a subsequent layer (e.g., the output layer 408). The dense layers 412 may be described as “dense” because each of the neurons in a given layer may receive an input from each neuron in a previous layer and provide an output to each neuron in a subsequent layer. The dense layers 412 may be followed by an output layer 408 that provides the outputs of the convolutional neural network. The outputs may be, for example, instructions to translate, rotate, and tilt an ultrasound imaging device. The output layer 408 may provide the outputs to translate, rotate, and tilt the ultrasound imaging device simultaneously and independently of each other. A processing device receiving the outputs from the output layer 408 may only choose to provide to a user one of these outputs at a time. For example, once the ultrasound imaging device is in a default orientation, the processing device may first provide translation instruction outputs from the neural network, then provide rotation instruction outputs from the neural network once there are no further translation instructions, and then provide tilt instruction outputs from the neural network once there are no further rotation instructions.

It should be appreciated that the convolutional neural network shown in FIG. 8 is only one example implementation and that other implementations may be employed. For example, one or more layers may be added to or removed from the convolutional neural network shown in FIG. 8. Additional example layers that may be added to the convolutional neural network include: a convolutional layer, a transpose convolutional layer, a locally connected layer, a fully connected layer, a rectified linear units (ReLU) layer, a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

For further description of deep learning techniques, see U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application), which is incorporated by reference herein in its entirety. In any of the embodiments described herein, instead of or in addition to using one or more convolutional neural networks, fully connected neural networks, random forests, support vector machines, linear classifiers, and/or other machine learning models may be used.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A method, comprising:
receiving, by a processing device, motion and/or orientation data from an ultrasound imaging device in operative communication with the processing device, wherein the motion and/or orientation data provides an indication of a motion and/or orientation of the ultrasound imaging device;
receiving, by the processing device, ultrasound data collected by the ultrasound imaging device; and
providing, by the processing device, an instruction for moving the ultrasound imaging device based on the motion and/or orientation data and the ultrasound data.

2. The method of claim 1, wherein the instruction for moving the ultrasound imaging device comprises an instruction for moving the ultrasound imaging device to a target position and orientation.

3. The method of claim 1, wherein the motion and/or orientation data and the ultrasound data are associated with each other based on time.

4. The method of claim 1, further comprising:
inputting the motion and/or orientation data and the ultrasound data to a statistical model configured to output the instruction for moving the ultrasound imaging device based on the motion and/or orientation data and the ultrasound data.

5. The method of claim 4, wherein:
the inputted motion and/or orientation data and the inputted ultrasound data comprise first motion and/or orientation data and first ultrasound data; and
the statistical model is configured to output the instruction for moving the ultrasound imaging device further based on second motion and/or orientation data and second ultrasound data, wherein the first motion and/or orientation data was received more recently than the second motion and/or orientation data and the first ultrasound data was received more recently than the second ultrasound data.

6. The method of claim 3, wherein the statistical model comprises a recurrent neural network.

7. The method of claim 6, wherein the recurrent neural network comprises a long short-term memory neural network.

8. The method of claim 1, wherein the ultrasound imaging device is configured to generate the motion and/or orientation data using one or more of an accelerometer, a gyroscope, or a magnetometer on the ultrasound imaging device.

9. An apparatus, comprising:
a processing device configured to:
receive motion and/or orientation data from an ultrasound imaging device in operative communication with the processing device, wherein the motion and/or orientation data provides an indication of a motion and/or orientation of the ultrasound imaging device;
receive ultrasound data collected by the ultrasound imaging device; and
provide an instruction for moving the ultrasound imaging device based on the motion and/or orientation data and the ultrasound data.

10. The apparatus of claim 9, wherein the instruction for moving the ultrasound imaging device comprises an instruction for moving the ultrasound imaging device to a target position and orientation.

11. The apparatus of claim 9, wherein the motion and/or orientation data and the ultrasound data are associated with each other based on time.

12. The apparatus of claim 9, wherein the processing device is further configured to:
input the motion and/or orientation data and the ultrasound data to a statistical model configured to output the instruction for moving the ultrasound imaging device based on the motion and/or orientation data and the ultrasound data.

13. The apparatus of claim 12, wherein:
the inputted motion and/or orientation data and the inputted ultrasound data comprise first motion and/or orientation data and first ultrasound data; and
the statistical model is configured to output the instruction for moving the ultrasound imaging device further based on second motion and/or orientation data and second ultrasound data, wherein the first motion and/or orientation data was received more recently than the second motion and/or orientation data and the first ultrasound data was received more recently than the second ultrasound data.

14. The apparatus of claim 11, wherein the statistical model comprises a recurrent neural network.

15. The apparatus of claim 14, wherein the recurrent neural network comprises a long short-term memory neural network.

16. The apparatus of claim 9, wherein the ultrasound imaging device is configured to generate the motion and/or orientation data using one or more of an accelerometer, a gyroscope, or a magnetometer on the ultrasound imaging device.

17. An apparatus, comprising:
a processing device configured to:
receive sets of ultrasound data two or more times from an ultrasound imaging device in operative communication with the processing device;
receive motion and/or orientation data from the ultrasound imaging device, wherein the motion and/or orientation data provides an indication of a motion and/or orientation of the ultrasound imaging device;
determine that the sets of ultrasound data and the motion and/or orientation data indicate a velocity of the ultrasound imaging device that exceeds a threshold velocity; and
based on determining that the sets of ultrasound data and the motion and/or orientation data indicate that the velocity of the ultrasound imaging device exceeds the threshold velocity, provide an instruction for slowing the velocity of the ultrasound imaging device.

18. The apparatus of claim 17, wherein the processing device is configured to access a statistical model configured to output the velocity of the ultrasound imaging device based on the sets of ultrasound data and the motion and/or orientation data.

19. The apparatus of claim 17, wherein the threshold velocity is related to a lag time between when the ultrasound imaging device collects ultrasound data and when the processing device provides an instruction for moving the ultrasound imaging device to a target location and orientation based on the ultrasound data.

20. The apparatus of claim 19, wherein the processing device is configured to access a statistical model configured to output the instruction for moving the ultrasound imaging device to the target location and orientation based on the ultrasound data.

21. The apparatus of claim 17, wherein the ultrasound imaging device is configured to generate the motion and/or orientation data using one or more of an accelerometer, a gyroscope, or a magnetometer on the ultrasound imaging device.

22. The apparatus of claim 17, wherein the threshold velocity is approximately in the range of 0.25 cm/s-2 cm/s.
